(19) United States
(12) Patent Application Publication
(10) Pub. No.: US 2008/0020061 A1
(43) Pub. Date: Jan. 24, 2008

(54) Therapeutic cream

(76) Inventors: Mark A. Hassler, Bethel, PA (US); Lisa M. Hassler, Bethel, PA (US)

Correspondence Address: Zachary T. Wobensmith, III, 7746 101st Court, Vero Beach, FL, 32967-2871, US

(21) Appl. No.: 11/801,432

(22) Filed: May 10, 2007

Related U.S. Application Data
(63) Continuation-in-part of application No. 11/490,364, filed on Jul. 21, 2006, now abandoned.

(51) Int. Cl. A61K 33/38

(52) U.S. Cl. 424/618

(57) ABSTRACT

Chemical compositions in cream form for the treatment of Bovine Mastitis in cows, which compositions are combinations of Aloe Vera, Colloidal Silver, Castor Oil, Glycosperse, Peppermint Oil, Peppermint Leaf, Tea Tree Oil, Oregano Oil, Poke Root, Vitamin C, Menthol crystals, Salt, Polysorbate 80 and Guar/Xanthan Gum, which provides a cream that is applied externally in one or more applications, to the affected portion of the cows udder, for the time required to provide a cure for the Mastitis.

### CROSS REFERENCE TO RELATED APPLICATIONS

This application is a Continuation in part of our prior application Ser. No. 11/490,364, filed on Jul. 21, 2006, entitled “Therapeutic Cream”.

### BACKGROUND OF THE INVENTION

1. Field of the Invention

This invention relates to therapeutic cream compositions applied externally to a cow's udder for treating Bovine Mastitis, which compositions include Aloe Vera, Colloidal Silver, Castor Oil, Glycosperse 020, Peppermint Oil, Peppermint Leaf, Tea Tree Oil, Oregano Oil, Poke Root, Vitamin C, Menthol crystals Salt, Polysorbate 80,and Guar/Xanthan Gum.

2. Description of the Prior Art

Dairy cows are bred and selected for their ability to produce large quantities of and high quality milk. Dairy cows are expensive to acquire and to maintain. Maximum milk production and minimum down time for the cows are very important in the dairy industry. Dairy cows are susceptible to a variety of ailments, which can affect their health and the production of wholesome milk, with Bovine Mastitis being a prime cause of milk production loss. If not cured of this condition, the cows may go to slaughter.

In our prior U.S. Pat. No. 5,846,543 chemical compositions are described which are intended to cure Bovine Mastitis. While the described compositions have proven highly satisfactory, they are intended to be injected directly into the cow's udder, which limits their use and makes administration more complicated than is desirable.

A cream that can be applied externally to the cow's udder by anyone, and which cures the inflammations or infections is highly desirable.

The application of the chemical compositions described herein has resulted in a complete cure of Bovine Mastitis in cows, for all the infected cows with which they have been used, with no deleterious effect on the cows' milk or noticeable side effects.

### SUMMARY OF THE INVENTION

This invention is directed to chemical compositions in cream form, for external application to a cow's udder, for the treatment of Bovine Mastitis, which compositions contain combinations of Aloe Vera, Colloidal silver, Castor oil, Glycosperse 020, Peppermint oil, Peppermint leaf, Tea Tree Oil, Oregano Oil, Poke Root, Vitamin C, Menthol crystals, Salt, Polysorbate 80, and Guar/Xanthan Gum, which are combined to provide a cream that is applied externally to the affected portion of the cow to be treated.

The principal object of the invention is to provide chemical compositions for treatment of Bovine Mastitis in cows.

A further object of the invention is to provide chemical compositions as aforesaid, which are safe and can be easily applied by anyone.

A further object of the invention is to provide chemical compositions as aforesaid, which are practical to use.

A further object of the invention is to provide chemical compositions as aforesaid, which are highly effective in treating Bovine Mastitis.

A further object of the invention is to provide chemical compositions as aforesaid, which do not produce undesirable side effects.

Other objects and advantageous feature of the invention will be apparent from the description and claims.

It should, of course, be understood that the description is merely illustrative and that various modifications and changes can be made in the compositions disclosed without departing from the spirit of the invention.

### DESCRIPTION OF THE PREFERRED EMBODIMENTS

When referring to the preferred embodiments, certain terminology will be utilized for the sake of clarity. Use of such terminology is intended to encompass not only the described embodiment, but also technical equivalents, which operate and function in substantially the same way to bring about the same result.

The described chemical compositions for treatment of Bovine Mastitis in cows, are formed of a number of components, in various concentrations, which are combined to provide the composition in cream form.

The first component is Aloe Vera juice at full strength. The juice contains 99.7% Aloe Vera and trace amounts of Potassium Sorbate, Sodium Benzoate, and Ascorbic Acid. Aloe Vera juice is available from Aloe Farms, Box 125, Los Fresnos, Tex. 78556.

The second component is Colloidal Silver in a water suspension.

The third component is Castor Oil.

The fourth component is Glycosperse

The fifth component is Peppermint Oil.

The sixth component is Peppermint Leaf.

The seventh component is Tea Tree Oil.

The eighth component is Oregano Oil.

The ninth component is Poke Root, available from Gaia Herbs, Inc. 12 Lancaster County Road, Harvard, Mass. 01451.

The tenth component is Vitamin C.

The eleventh component is Menthol crystals

The twelfth component is Guar/Xanthan Gum.

The thirteenth component is salt.

The fourteenth component is Polysorbate 80.

The fourteen components are combined in the following % of volume ranges to form a batch of the chemical compositions in cream form.

Aloe Vera in the range of 28 to 90%.

Colloidal Silver in a water suspension (1 to 2000 ppm) in the range of 30 to 70%.

Castor Oil in the range of 4% to 96%.

Glycoperse in the range of 6% to 96%.

Peppermint Oil in the range of greater than zero to 4%.

Peppermint Leaf in the range of greater than zero to 10%.

Tea Tree Oil in the range of greater than zero to 16%.

Oregano Oil in the range of greater than zero to 4%.

Poke Root in the range of greater than zero to 8%.

Vitamin C in the range of greater than zero to 2.4% (200 IU).

Menthol crystals in the range of greater than zero to 2.4%.

Guar/Xanthan Gum in the range of greater than zero to 24%.

Salt in the range of greater than zero to 2.4%.

Polysorbate 80 in the range of greater than zero to 2.4%.

A preferred composition for a 500 ml batch is two cups of thickened Aloe Vera, two cups of Colloidal Silver in a water suspension (11 ppm), eight ounces of Castor Oil, eight ounces of Glycosperse, six milliliters of Peppermint Oil, ten milliliters of Peppermint Leaf, six milliliters of Tea Tree Oil, ¾ milliliter of Oregano Oil, five milliliters of Poke Root, one tablespoon Vitamin C (200 iu), one ounce of Menthol crystals, four ounces of Salt, four ounces of Polysorbate 80, and two and a half pounds of Guar/Xanthan Gum, as required to achieve the desired consistency for cream application.

The cream has been applied to the udders of dairy cows that exhibited a Somatic cell count (SCC) in excess of 750,000, which is indicative of Bovine Mastitis and which count is the maximum allowable count without treatment, and positive results were obtained as follows

### EXAMPLE #1

### Cow #13:

Somatic cell count (SCC): was initially 5,500,000. The cream as described was applied directly onto the cow's udder just after milking, and continued applications for one week. After five milkings the SCC had dropped to 1,300,000. After four more milkings over two days, the SCC was 475,000. The cream application was continued, and after four more milkings over two days, the SCC was 150,000. Cream application was stopped and after two milkings the SCC was 94,000. After eight more milkings, SCC was 95,000, and after eight additional milkings, the SCC was 92,000. After a two-month period from the first cream application, the SCC was 91,000.

### EXAMPLE #2

### Cow #46:

The initial SCC was 874,000. The cream as described was applied onto the cow's udder for five milkings. The SCC was tested and was 210,000. After four additional milkings the SCC was 78,000. After two months, the SCC was 67,000.

### EXAMPLE #3

### Cow #47

The initial SCC was 8,400,000. The cream as described was applied onto the cow's udder, and applications continued for five milkings when the SCC was 5,000,000. The cream applications were continued and after four more milkings the SCC was 470,000. The cream applications were continued for four more days but the SCC was not checked. One month after the first cream application, the SCC was checked and was 205,000.

It will thus be seen that compositions for the treatment of Bovine Mastitis in cows have been provided with which the objects of the invention are achieved.

1. A composition for the treatment of Bovine Mastitis in cows,
comprising,
Aloe Vera juice,
Colloidal Silver,
Castor Oil,
Glycosperse,
Peppermint Oil,
Peppermint Leaf,
Tea Tree Oil,
Oregano Oil,
Poke Root,
Vitamin C,
Menthol crystals,
Guar/Xanthan Gum,
Salt, and
Polysorbate 80.

2. The composition as defined in claim 1 in which,
said Aloe Vera juice is in a range of 28% to 90% by volume.

3. The composition as defined in claim 1 in which,
said Colloidal Silver is (1 to 2000 ppm) in a water suspension which is of 30% to 70% by volume.

4. The composition as defined in claim 1 in which,
said Castor Oil is in a range of 4% to 96% by volume.

5. The composition as defined in claim 1 in which,
said Glycosperse is in a range of 6% to 96% by volume.

6. The composition as defined in claim 1 in which,
said Peppermint Oil is in a range of greater than 0% to 4% by volume.

7. The composition as defined in claim 1 in which,
said Peppermint Leaf is in a range of greater than 0% to 10% by volume.

8. The composition as defined in claim 1 in which,
said Tea Tree Oil is in a range of greater than 0% to 16% by volume.

9. The composition as defined in claim 1 in which,
said Oregano Oil is in a range of greater than 0% to 4% by volume.

10. The composition as defined in claim 1 in which,
said Poke Root is in a range of greater than 0% to 8% by volume.

11. The composition as defined in claim 1 in which,
said Vitamin C is in a range of greater than 0% to 2.4% by volume.

12. The composition as defined in claim 1 in which,
said Menthol crystals are in a range of greater than 0% to 2.4% by volume.

13. The composition as defined in claim 1 in which,
said Guar/Xanthan Gum is in a range of 0% to 24% by volume.

14. The composition as defined in claim 1 in which,
said salt is in a range of greater than 0% to 2.4% by volume.

15. The composition as defined in claim 1 in which
said Polysorbate 80 is in a range of greater than 0% to 2.4% by volume. A composition for the treatment of Bovine Mastitis in cows consisting essentially of,
Aloe Vera juice,
Colloidal Silver,
Castor Oil,
Glycosperse,
Peppermint Oil,
Peppermint Leaf,
Tea Tree Oil,
Oregano Oil
Poke Root,
Vitamin C
Menthol crystals,
Guar/Xanthan Gum
Salt, and
Polysorbate 80.

16. A method of treating Bovine Mastitis in cows comprising the steps of
providing a composition for the treatment of Bovine Mastitis,
said composition comprising,
Aloe Vera juice,
Colloidal Silver,
Castor Oil,
Glycosperse,
Peppermint Oil,
Peppermint Leaf,
Tea Tree Oil,
Oregano Oil,
Poke Root,
Vitamin C,
Menthol crystals,
Guar/Xanthan Gum,
Salt, and
Polysorbate 80,
administering an effective dosage of the composition to the cow by externally applying it to an affected portion of an affected cow's udder, and continuing to apply additional effective amounts of the composition periodically throughout a treatment period.
